(19) United States
(12) Patent Application Publication
(10) Pub. No.: US 2026/0166247 A1
(43) Pub. Date: Jun. 18, 2026

(54) A METHOD OF PROTECTING TURBINE SHAFT BEARINGS USED IN ANAESTHESIA DEVICES FROM CONTAMINANTS IN PATIENT GASES

(71) Applicant: Peninsula Medical Technologies Ltd, Totnes, Devon (GB)

(72) Inventor: Sebastian BROWN, Totnes, Devon (GB)

(21) Appl. No.: 19/126,804

(22) PCT Filed: Nov. 2, 2023
(86) PCT No.: PCT/EP2023/080616

§ 371 Date: May 2, 2025

(30) Foreign Application Priority Data
Nov. 3, 2022 (GB) 2216366.1
Mar. 15, 2023 (GB) 2303766.6

(51) Int. Cl. A61M 16/00; A61M 16/01

(52) U.S. Cl. CPC A61M 16/0066; A61M 16/0093; A61M 16/01; A61M 2202/0208; A61M 2205/3334

(57) ABSTRACT

A medical equipment turbine is provided for the delivery of gas to a patient. The turbine comprises motor and a housing including a motor shaft (7) having a motor end. The turbine further comprises a gas channel (6) to the motor-end of the shaft, whereby flushing gas can be delivered to the region of the base of the motor shaft and distributed by a collar (5) to prevent exposure of the motor bearings to patient gases. A method for the prevention of exposure of bearings and lubricants in turbine motors used in medical ventilators and anaesthesia devices is also disclosed, where a flow of medical gas is delivered to the base of the motor shaft and distributed by a collar surrounding the shaft to prevent the exposure of the motor bearings to patient gases and/or volatile anaesthetic.

Modern ventilation systems used to replace or assist breathing function for patients undergoing surgery or who are unable to breathe normally for themselves due to illness often use turbines (“blowers”) for the delivery of pressure to the patient. Turbines consist of a motor, impeller, gas casing and heatsink. Gas is drawn in through the inlet and passes out through the outlet. There are different configurations of turbine (single stage, dual stage etc.) and the turbine can be used in different ways; highly dynamic turbines can speed up and down in sequence with the breath and less dynamic turbines may provide a high pressure to a valve that allows gas in at the desired flow rate for the breath.

The present invention refers in part to the use of turbines in a modular anaesthesia and ventilation system described in WO 2022/049389, the contents of which are hereby incorporated by reference. In this system, a portable ventilator module provides ventilation for patients in transport, intensive care, wards and long-term ventilation facilities. Ventilation can be invasive ventilation, non-invasive ventilation or the provision of high flow oxygen in mandatory, spontaneous or mixed modes as familiar to those skilled in the art. When working alone, the ventilation module mixes pressurised oxygen with ambient air drawn into the device by the turbine as determined by the user. This gas mixture is then delivered to the patient during the inspiratory phase and subsequently exhaled, through a valve that maintains the circuit pressure, to atmosphere. At each point in the circuit, additional components are required to monitor and control pressure, temperature and flow.

The portable ventilation module can be reversibly connected (“docked”) to an anaesthesia module to provide functionality required for anaesthesia. This includes the addition and recirculation of anaesthetic agent (volatile anaesthetics such as Sevoflurane, Desflurane or Isoflurane or gases such as xenon or nitrous oxide), addition of oxygen/air, removal of carbon dioxide, provision for manual ventilation, exhaust and waste gas scavenging systems. Other systems may be included to control temperature and/or humidity.

As anaesthesia systems recirculate exhaled patient gas, these systems must be suitable for cleaning/sterilisation as required and compatible with anaesthetic agents/gases used to induce or maintain anaesthesia. These anaesthesia gases (as mentioned above) are effective solvents for many standard lubricants or incompatible with often-used seals (e.g. FKM). Due to the high speed of rotation of the turbine (e.g. 60,000 rpm) and dynamic nature (the turbine may accelerate and decelerate up to 100 times per minute for each breath), it is challenging to seal the turbine shaft to prevent exposure of the shaft bearings and lubricant to gases passing through the turbine. These gases contain anaesthetic vapour and microbiological contamination from exhaled patient gases and can affect the long-term performance of the turbine by degrading the lubricant or contaminate the turbine bearings/shaft which may then not sterilisable.

Solutions that may remove the impeller from the turbine motor for cleaning/sterilisation are not favourable as the high impeller rotational speed needs very precise balancing after manufacture. Removal and replacement may affect this balance leading to damage to the turbine motor and degradation in performance during the operation of the turbine.

The present invention relates to apparatus and methods to prevent the impairment of performance of lubricants used in the motor by exposure to volatile anaesthetic gases used in anaesthesia. This may be provided by a modular system as mentioned above or by conventional anaesthesia systems incorporating a turbine.

### DESCRIPTION OF INVENTION

The present invention provides or relates to apparatus and a method resulting in the protection of turbine shaft lubricants and components (e.g. bearings) from degradation induced by anaesthesia gases (Sevoflurane, Desflurane, Isoflurane, Nitrous Oxide and Xenon) by using a flush gas flow of pure oxygen through a gas collar surrounding the turbine shaft.

The gas collar serves to deliver the oxygen gas to the shaft and reduce the outward flow of oxygen gas to maintain a minimal gas flush flow rate. As oxygen is always required by the patient as a basal metabolic rate and a basal flow of oxygen is recommended/required by standards/convention, this gas flush does not generate additional wastage of gas or impair the delivery of anaesthesia, especially when anaesthesia gas injection systems are used familiar to those skilled in the art.

In one embodiment of the invention, a gas channel is made from the outside of the turbine to the motor-end of the shaft. A connection is made at the external end of this gas channel for the connection of the oxygen supply.

A collar may be secured to the turbine housing, around the shaft. This collar may be a separate component sealed to the turbine housing as a separate component or integrated as part of the turbine housing design. The collar serves to distribute gas from the channel around the base of the motor shaft. This gas then passes around the shaft and underneath the impeller to join the gas from the breathing circuit.

In this way, at outward flow of gas from the shaft is provided to prevent patient gas containing anaesthesia gas and contaminants from surrounding the bearing and interfering with the function of the bearing.

In one embodiment, the collar is a separate piece that is secured to the motor or turbine housing by screws and may contain a seal to prevent gas from flowing between the collar and the housing.

In another embodiment, the turbine housing is built with the collar integrated into the housing.

In one embodiment, the collar is made of metal, e.g. aluminium or stainless steel or another suitable metal familiar to those skilled in the art.

In another embodiment, the collar is made of plastic such as nylon, polyetherimide (PEI) or PEEK or another suitable plastic familiar to those skilled in the art.

In one embodiment as shown in the drawings, the collar does not extend to the full diameter of the impeller.

In another embodiment, the collar extends to the full diameter of the impeller.

In one embodiment, there is a single gas channel.

In another embodiment, there are multiple gas channels.

In one embodiment, the impeller-side of the collar has a smooth surface.

In another embodiment, the impeller-side of the collar has a surface with recessed circles of increasing diameter to trap gas and form a gas-seal with the underside of the impeller.

In a preferred embodiment, the internal surface of the collar has grooves machined into the face that is in proximity to the shaft that are filled with flush gas and operate as a gas bearing on the shaft, preventing ingress of gases contaminated with anaesthetic vapours or exhaled from the patient (and containing microbiological contaminants).

In one aspect of the invention, a plate is recessed into the heatsink to maintain a flat surface and acts as a distributor of gas from the oxygen connection source to the motor shaft. The plate is secured by screws and sealed at the distal edge by o-ring seal to direct gas to the motor shaft, although other methods of securing and sealing such as adhesive or sealant are envisaged.

In one embodiment of the invention, a gas collar is part of the distributor plate to further protect the motor shaft from the external volatile anaesthetic agent.

In one aspect of the invention, the flow rate of flush gas is varied according to the concentration and type of volatile anaesthetic agent being used. The volatile anaesthetic agents may be used in different concentrations depending on the depth of anaesthesia required or the type of anaesthetic in use. Thus, to achieve the same depth of anaesthesia, Desflurane needs to be given at concentrations of around 6% (one Minimum Alveolar Concentration—MAC) and Sevoflurane at 2%. In one embodiment of the invention, the flush gas rate is increased when used with higher concentrations of anaesthetic gas.

In another embodiment of the invention, the flush gas rate is controlled by a computer model based on the concentration and type of anaesthetic gas.

In a further embodiment of the invention, the flush gas rate is controlled by a computer model based on the concentration and/or type of anaesthetic gas and/or rotational speed of the turbine.

Activated carbon is an effective absorbent of volatile anaesthetic agents. Depending on the carbon source, pore size and dopants, the binding affinity can be adjusted. The use of a flush gas will drive absorbed volatile anaesthetic from the activated carbon.

In an aspect of the invention, a replaceable activated carbon collar is placed around the motor shaft. This activated carbon collar provides a physical partial barrier to the passage of anaesthetic gases to the motor shaft. It also absorbs any volatile anaesthetic agent near the motor shaft. Due to the presence of the flush gas which is delivered to the motor side of the activated carbon and flows around/through the activated carbon to pass to the turbine outlet, any absorbed volatile anaesthetic is desorbed from the activated carbon, further protecting the bearings/lubricants from degradation by exposure to volatile anaesthetic gases.

In an aspect of the invention, the flush gas may be pure oxygen delivered to the turbine within the machine. This may be medical oxygen in the case of a medical device. This may be medical air if oxygen is not required. This may be a mixture of medical oxygen/medical air as required.

In an aspect of the invention, the flush gas may be recycled from the patient circuit. Patient gas is withdrawn from the patient circuit by a pump, as familiar to those skilled in the art. The flush gas may then be scrubbed of moisture by the use of absorbent materials (e.g. silica gel) or a membrane (e.g. Nafion) and. The gas is then scrubbed of volatile anaesthetic agent by a carbon filter before being passed back into the turbine as flush gas with no/low concentration of volatile anaesthetic gas.

In one embodiment of the invention, the moisture scrubbing absorbent is replaceable.

In another embodiment of the invention, the carbon filter is replaceable.

In a preferred embodiment of the invention, two carbon filters are used. In one state, patient gas is passed into a first carbon filter where volatile anaesthetic is absorbed onto the filter and then to the turbine as a flush gas. At the same time, fresh oxygen and/or air is passed through a second carbon filter to remove absorbed volatile anaesthetic, regenerate the filter, and return the anaesthetic to the anaesthesia circuit. When the first carbon filter is nearly saturated, the state is changed so that the first carbon filter is exposed to fresh oxygen and/or air to remove absorbed volatile anaesthetic agent, regenerate the filter, and return the volatile anaesthetic agent to the patient circuit and patient gas is passed into the second carbon filter to remove volatile anaesthetic agent, which is then used as a flush gas for the turbine.

In one embodiment of the invention, the filter material may be one of activated carbon, silica, halogenated carbon, carbon, graphite, aerogel (carbon or silica-based) or other filter materials known to reversibly absorb volatile anaesthestics/fluorocarbons to those skilled in the art.

In one embodiment of the invention, the canister may be heated or exposed to vacuum during regeneration to improve the desorption of volatile anaesthetic.

In one embodiment of the invention, water may be removed before the absorption of volatile anaesthetic, by the use of a preceding water-absorbent filter such as silica gel. Due to the presence of exhaled patient gas in circle systems used for anaesthesia gas delivery and efficiency, the flush gas also prevents contamination of the turbine shaft/bearings with exhaled patient gas which may contain chemical or microbial contaminants (e.g. viruses/spores/bacteria etc). The presence of exhaled patient gas in the circuit means that the areas of the turbine exposed to patient gas must be capable of sterilisation or disinfection.

In one embodiment of the invention, the impeller housing is detachable from the turbine heat sink, motor, impeller and flush gas mechanism. The impeller housing can then be taken away by the user for sterilisation (e.g. autoclave although other methods as mentioned below or known to those skilled in the art can be used) and replaced when cleaned. By only removing the impeller housing, the flush gas mechanism can remain connected to the turbine. As this is a gas connection, it is preferable to leave this connection intact rather than it being repeatedly broken and re-made for each sterilisation cycle.

In an embodiment of the invention, the impeller housing may be replaced by another housing that is able to administer a sterilising agent to the remaining components of the turbine so that these may also be sterilised or disinfected in situ. Such sterilising agents may include but are not limited to ethylene oxide, hydrogen peroxide (vapourised or plasma) or chlorine dioxide although other methods are familiar to those skilled in the art.

In one embodiment of the invention, the replacement housing for sterilising the remaining parts of the turbine introduces a source of UVC radiation that is configured to be delivered to all parts of the turbine that are exposed to exhaled patient gas.

In a preferred embodiment, the use of the flush gas delivered to the shaft of the turbine means that this is not exposed to exhaled patient gas and only the parts after the exit of flush gas from the collar are considered exposed, and these are accessible to the UVC radiation for sterilisation.

In another embodiment of the invention, other direct sources of sterilisation may be used in place of or in addition to UVC radiation. These include gamma irradiation and electron beam irradiation, although other methods may be used familiar to those skilled in the art.

Each means of sterilisation can be controlled by a device attached to or as part of the sterilising impeller housing or the sterilisation process may be controlled by the ventilator module itself following electrical (communications and/or power) connection to the sterilising impeller housing.

Further aspects and embodiments are provided in the following numbered paragraphs.
1. A turbine for the delivery of gas to a patient for ventilation whereby flushing gas can be delivered to the region of the base of the motor shaft to prevent exposure of the motor and bearings to patient gases or gases containing volatile anaesthetic.
2. The turbine of claim 1, in which a gas collar is configured to deliver the flushing gas to the motor end of the shaft and whereby flushing gas passes between the collar and shaft towards the impeller end of the shaft to maintain a minimal gas flush flow rate.
3. The turbine of claim 1 or claim 2, whereby a connection can be made at the external end of the gas channel for the connection of a flushing gas supply.
4. The turbine of any preceding claim, in which the collar is a separate component that is secured to the turbine housing, around the shaft.
5. The turbine of any of claims 1 to 3, in which the collar is integrated as part of the turbine housing.
6. The turbine of any preceding paragraph, in which the collar is configured such that gas then passes around the shaft and underneath an impeller to join gas from a breathing circuit.
7. The turbine of any preceding paragraph, in which the collar does not extend to the full diameter of an impeller or in which the collar extends to the full diameter of an impeller.
8. The turbine of any preceding paragraph, in which the collar has a single gas channel or multiple gas channels.
9. The turbine of any preceding paragraph, in which a motor shaft surface of the collar is in close proximity to the motor shaft and has recessed circles in the collar to trap gas and form a gas-bearing seal with the motor shaft.
10 The turbine of any preceding paragraph, in which an impeller-side of the collar has a surface with recessed circles of increasing diameter to trap flushing gas and form a gas-seal with the underside of the impeller.
11. A medical equipment turbine for the delivery of gas to a patient, the turbine that has a flow of medical oxygen delivered to the base of the motor shaft and distributed by a collar surrounding the shaft to prevent the exposure of the motor bearings to inspiratory and/or expiratory patient gases.
12. A turbine in any preceding paragraph which has a removable impeller housing that can be sent for sterilisation and wherein the turbine housing, motor and impeller remain in the device to maintain electrical and flush gas connections.
13. A turbine in paragraph 13 wherein the removable impeller housing can be replaced by a sterilisation device that delivers sterilising fluids, vapours or radiation to the turbine housing, motor and impeller to sterilise the parts of the turbine remaining in the device after the impeller housing is removed.
14. A turbine of any preceding paragraph whereby the flushing gas is medical oxygen or medical air.
15. A patient anaesthesia device having a turbine of any preceding paragraph.
16. A method for the prevention of exposure of bearings and lubricants in turbine motors used in medical ventilators and anaesthesia devices where a flow of medical gas is delivered to the base of the motor shaft and distributed by a collar surrounding the shaft to prevent the exposure of the motor bearings to patient gases and/or volatile anaesthetic.
17. A method for the prevention of exposure of bearings and lubricants in turbine motors used in medical ventilators and anaesthesia devices wherein a collar made of a porous filter material that reversibly absorbs volatile anaesthetic is situated below the impeller in proximity to the motor shaft and whereby a flow of clean gas is delivered to the inside of the collar to prevent the ingress of volatile anaesthetic to the motor bearings and lubricant.
18. A method for the prevention of exposure of bearings and lubricants in turbine motors used in medical ventilators and anaesthesia devices as described in paragraph 14 wherein the clean gas is provided by removing gas from the patient circuit and scrubbing it of water and/or volatile anaesthetic agent by the use of selective filter materials prior to delivery to the turbine.
19. A turbine-based system for the delivery of gas to a patient, in which flushing gas is delivered to the turbine motor shaft to prevent exposure of the motor to patient gases or gases containing volatile anaesthetic.

Different aspects and embodiments of the invention may be used separately or together.

Further particular and preferred aspects of the present invention are set out in the accompanying independent and dependent claims. Features of the dependent claims may be combined with the features of the independent claims as appropriate, and in combination other than those explicitly set out in the claims.

The present invention is now more particularly described, by way of example, with reference to the accompanying drawings.

Example embodiments are described in sufficient detail to enable those of ordinary skill in the art to embody and implement the systems and processes herein described. It is important to understand that embodiments can be provided in many alternate forms and should not be construed as limited to the examples set forth herein.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

### DESCRIPTION OF DRAWINGS

FIG. 1 shows the turbine heatsink 2, cover 1, impeller 3 and motor 7 in cross section. A plate 5 is secured to the heatsink with screws. The plate conceals a channel 6 for gas to flow towards the motor spindle 7. Flush gas is contained at the outer edge by an o-ring seal 4 to ensure that gas exits the channel 6 by the motor spindle 7.

FIG. 2 shows the channel through the heatsink 11 that passes flush gas to the channel 6 in FIG. 1. A thread 12 may be cut into the channel to secure a connection (not shown) to the heatsink.

FIG. 3 shows the plate with a raised collar 22 that comes in close proximity to the impeller base to further direct gas to the spindle of the motor through the channel 21.

FIG. 4 shows an activated carbon filter 31 in close proximity to the impeller. The carbon filter rests on the heatsink but has ridges/channels on the underside to allow the passage of gas towards the spindle.

FIG. 5 shows a 1^{st} and 2^{nd} activated charcoal canister connected to patient circuit and medical gas inlets and to patient circuit and turbine flush outlets. At any stage, by the use of 3 port, 2 position valves, one carbon canister is scrubbing gas from the patient circuit and one carbon canister is being regenerated by a flow of clean medical gas through the canister and back into the patient circuit.

In (5A), canister 1, filled with absorbent, is used to scrub patient gases for use as the turbine flush while canister 2 may be emptied of anaesthetic by the medical gas flush back into the patient circuit to prevent loss of anaesthetic.

In (5B), canister 2 is used to scrub patient gases for use as the turbine flush and canister 1, as this is now full of absorbed anaesthetic, is emptied of anaesthetic back into the patient circuit.

It is envisaged that a circuit configuration can include adsorption and desorption steps with gas flow in the opposite direction to improve efficiency.

FIG. 6 shows the turbine 61 engaged into the patient circuit manifold 62.

FIG. 7 shows the turbine heat sink, motor and impeller 71 separated from the patient circuit manifold 72. The turbine heat sink, motor and impeller are left behind in the device and remain electrically connected and connected to the flush gas source in the device. The patient circuit manifold, which includes the impeller housing 73 can then be taken away for disassembly and sterilisation while the turbine heat sink, motor and impeller remain for in-situ sterilisation as described in the description.

FIG. 8a/8b are a ventilation turbine without the impeller housing/cover, showing the channel and collar for the supply of flush gas in side view (8a) and mid-section (8b). The heatsink and motor 81 are attached to the collar 83 which supplies flush gas from the gas inlet port 84 to the base of the shaft 85 to the impeller 82. This collar directs the gas around the shaft to protect the bearings from contamination, with gas flowing radially outwards from the shaft 85, under the impeller 82 to the outlet of the turbine (not shown). This prevents contamination of the turbine bearings with anaesthetic gases or exhaled patient gases.

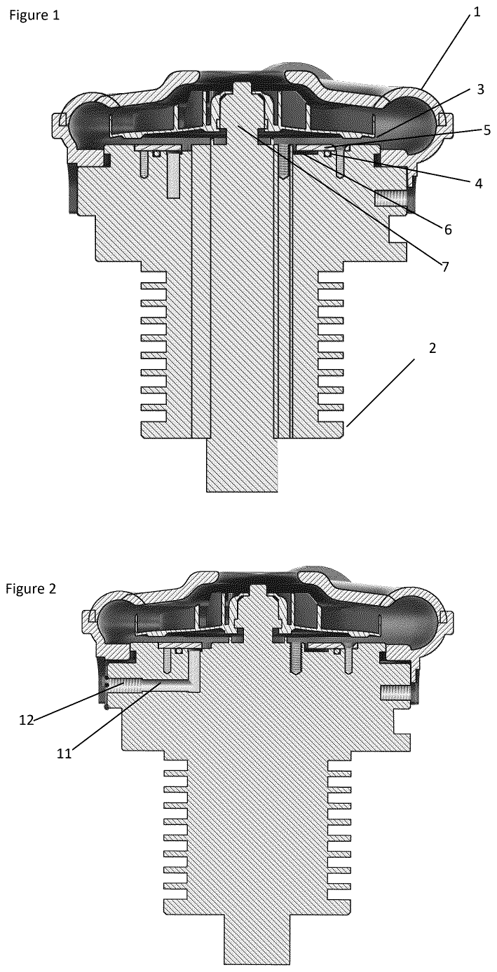

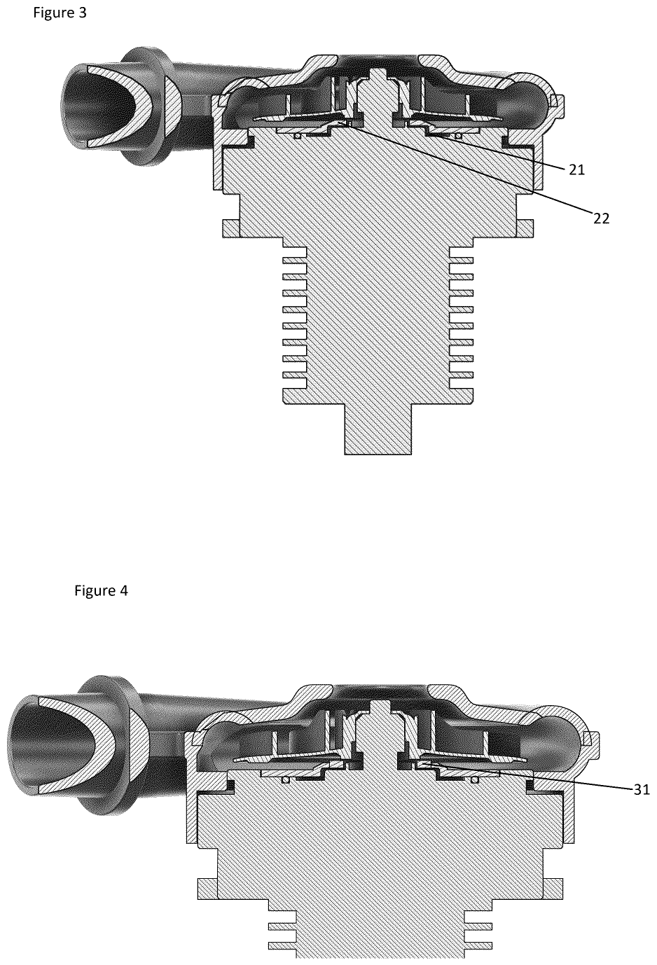

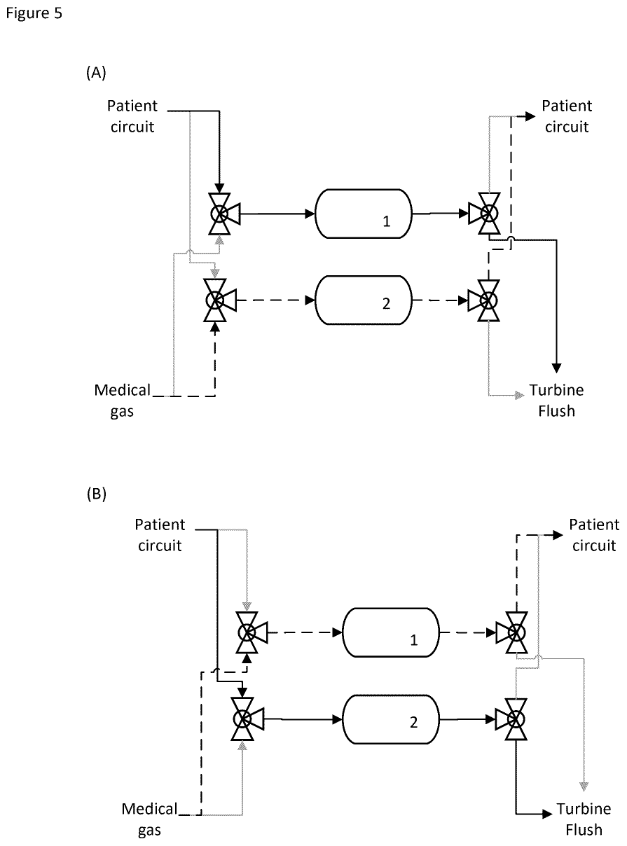

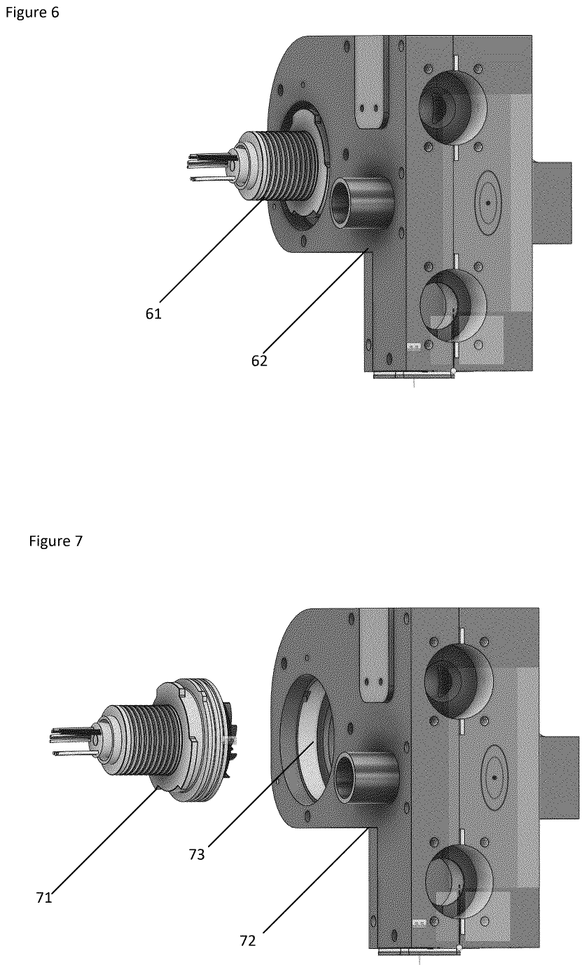

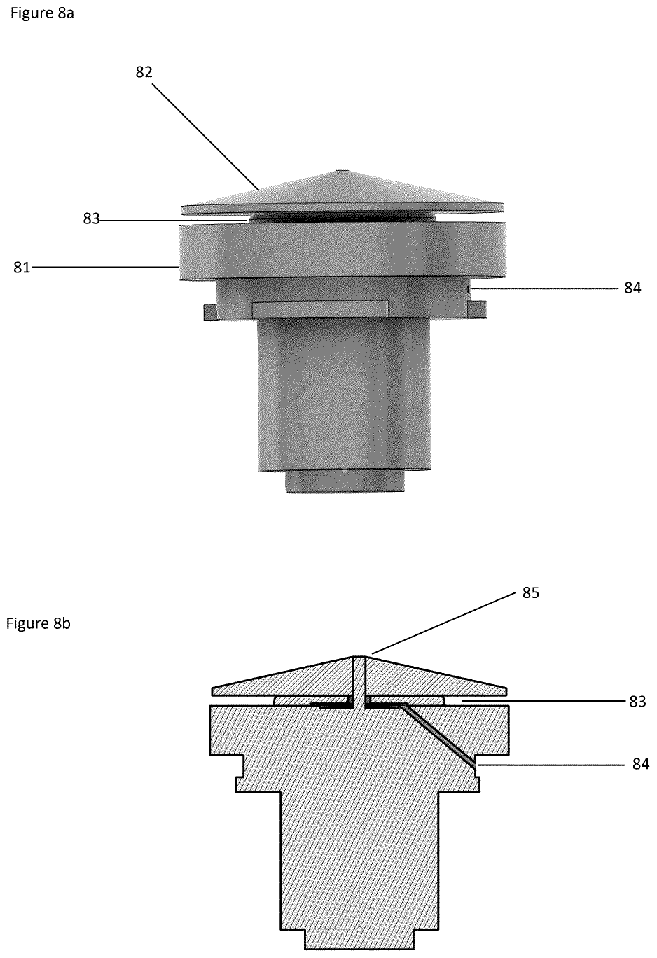

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention.

1. A turbine for the delivery of gas to a patient, the turbine comprises a motor and a housing including a motor shaft having a motor end, the turbine further comprises a gas channel to the motor-end of the shaft, whereby flushing gas can be delivered to the region of the base of the motor shaft and distributed to prevent exposure of the motor to patient gases.

2. A turbine according to claim 1, in which a collar is configured to deliver the flushing gas to the shaft and reduce the outward flow of oxygen gas to maintain a minimal gas flush flow rate.

3. A turbine according to claim 2, in which flushing gas passes between the collar and shaft towards an impeller end of the shaft to maintain a minimal gas flush flow rate.

4. A turbine according to claim 1, whereby a connection can be made at the external end of the gas channel for the connection of flushing gas supply.

5. A turbine according to claim 1, in which the collar is a separate component that is secured to the turbine housing, around the shaft.

6. A turbine according to claim 1, in which the collar is integrated as part of the turbine housing.

7. A turbine according to claim 1, in which a collar is configured such that flushing gas then passes around the shaft and underneath an impeller to join gas from a breathing circuit.

8. A turbine according to claim 1, in which a gas channel collar does not extend to the full diameter of an impeller, or in which the collar extends to the full diameter of an impeller.

9. A turbine according to claim 1, in which a collar has a single gas channel or multiple gas channels.

10. A turbine according to claim 1, in which a motor shaft surface of the collar is in close proximity to the motor shaft and has recessed circles in the collar to trap gas and form a gas-bearing seal with the motor shaft.

11. A turbine according to claim 1, in which an impeller-side of the collar has a smooth surface.

12. A turbine according to claim 1, in which an impeller-side of the collar has a surface with recessed circles of increasing diameter to trap flushing gas and form a gas-seal with the underside of the impeller.

13. A turbine according to claim 1 which has a removable impeller housing that can be sent for sterilisation and wherein the turbine housing, motor and impeller remain in the device to maintain electrical and flush gas connections.

14. A turbine-as according to claim 13, wherein the removable impeller housing can be replaced by a sterilisation device that delivers sterilising fluids, vapours or radiation to the turbine housing, motor and impeller to sterilise the parts of the turbine remaining in the device after the impeller housing is removed.

15. A turbine according to claim 1, in which the flushing gas is medical air and/or medical oxygen.

16. (canceled)

17. (canceled)

18. A turbine for the delivery of gas to a patient for ventilation that has a flow of medical oxygen delivered to the base of the motor shaft and distributed by a collar surrounding the shaft to prevent the exposure of the motor bearings to patient gases.

19. A method for the prevention of exposure of bearings and lubricants in turbine motors used in medical ventilators and anaesthesia devices where a flow of medical gas is delivered to the base of the motor shaft and distributed by a collar surrounding the shaft to prevent the exposure of the motor bearings to patient gases and/ or volatile anaesthetic.

20. A turbine according to claim 1, wherein a collar made of a porous filter material that reversibly absorbs volatile anaesthetic is situated below an impeller in proximity to a motor shaft and whereby a flow of clean gas is delivered to the inside of the collar to prevent the ingress of volatile anaesthetic to the motor bearings and lubricant.

21. A turbine according to claim 20, wherein the clean gas is provided by removing gas from the patient circuit and scrubbing it of water and/or volatile anaesthetic agent by the use of selective filter materials prior to delivery to the turbine.
